(19) United States
(12) Patent Application Publication
(10) Pub. No.: US 2011/0172628 A1
(43) Pub. Date: Jul. 14, 2011

(54) Leg And Waist Band Structures For An Absorbent Article

(76) Inventors: Donald Carroll Roe, West Chester, OH (US); Elaine Mary Wiggins, Fairfield Township, OH (US)

(21) Appl. No.: 12/687,425

(22) Filed: Jan. 14, 2010

(51) Int. Cl. A61F 13/45; B32B 3/06

(52) U.S. Cl. 604/385.29; 428/101

(57) ABSTRACT

An elasticized structure suitable for use as a legband or waistband in a wearable article. The elasticized structure includes an inelastic sheath and an elastic component bonded at least three points. The bond points are selected to influence aesthetics, force distribution, and gathering of the inelastic sheath about the elastic component.

### FIELD OF THE INVENTION

The disclosure relates generally to legband and waistband structures useful in reusable apparel articles, and more specifically to legband and waistband structures useful in reusable outer covers for absorbent articles, such as outer covers for diapers, training pants, adult incontinence devices, catamenial products, and the like.

### BACKGROUND OF THE INVENTION

Elastic structures are known for use to provide form-fitting apparel for a range of user sizes and shapes. Elastic structures may also be used to improve the fit and containment provided by absorbent articles, such as diapers, training pants, adult incontinence devices, catamenial products, and the like. Two basic elastic structures are common, each generally associated with a particular kind of article.

For disposable articles, such as disposable diapers, an elastic component may be extended, joined to another, possibly non-elastic, component of the disposable article in a non-extended state, and then allowed to relax. The result is an elastic structure that gathers the non-elastic component and provides stretch to the composite so formed. The composite can then be used to form, for example, a leg band or a waist band. The added elasticity may improve fit, and, in the case of disposable absorbent articles, may reduce leakage of exudates from the article as a result of the gasketing force of the elastic component against the wearer's body. Such elasticized composites, including elements such as elastic ribbons, yarns, films, etc., may be included, for example, in the leg and/or waist of the article.

In a disposable article, the elastic may be bonded to the non-elastic component along the entire length of the elastic. However, if the non-elastic component is bonded along all or substantially all of the length of the elastic, the elastic may be more or less contracted in various localized regions of the leg or waist hoop, leading to gapping or skin marking from low or high force areas. Accordingly, the bond between the elastic and non-elastic component or components may be either weak enough to permit the non-elastic material to pull away from the elastic as it gathers, or, an intermittent bond (e.g., using spiral adhesive application patterns with gaps between bonding locations) to provide space for the non-elastic material to gather after the elastic is relaxed. Intermittent bonding may not be adequate to produce a reusable or washable article. For example, adhesive spot bonds may not provide adequate securement to maintain the composite in its intended configuration under the stresses of multiple machine washing cycles. Further, the relationship between the elastic and non-elastic components is pre-determined and cannot be adjusted during wear, as to accommodate variations in the sizes and shapes of different wearers.

For apparel, such as underwear made of cotton or other fabrics, an elastic component may be encased in a tubular sheath. The sheath may be formed of the same material as the article itself. For example, the sheath may be formed by folding a length of the fabric of the article back onto itself, and stitching the inboard edge of the folded fabric down to form a tube. The elastic may be present within the sheath when the sheath is formed, or fed into the sheath after the sheath is formed. The ends of the elastic component are secured, often by stitching the ends of the elastic component into one of the seams in the article. The elastic can then extend and contract within the sheath. Alternately, the entire length of the elastic component may be stitched into the material at or near the edge of the article in an extended state, such that the material along the elastic component is gathered when the elastic component is allowed to relax.

An elastic component encased in a sheath may provide a robust structure which will survive many cycles of machine washing. An elastic component encased in a sheath may also provide an aesthetically pleasing effect, where the sheath matches or coordinates with the main body of the article. For example, the sheath may be the same color and type of fabric as the main body of the article, or may be a contrasting color and type of fabric selected for decorative effect. However, where the elastic can move freely in the sheath, the material of the sheath can shift and bunch, which may create uncomfortable bulk or even pinch points at localized areas of the leg band or waist band. Where the elastic is secured along the entire length of the leg band or waist band, the relationship between the elastic and non-elastic components is pre-determined and cannot be adjusted during wear, as to accommodate variations in the sizes and shapes of different wearers. A common alternative to a fixed elastic structure is a drawstring structure, wherein an elastic or non-elastic component is fed through a sheath, and can be moved relative to the sheath to adjust the size of the leg or waist opening. However, a drawstring structure, using either an elastic drawstring or an inelastic drawstring, suffers from the same problems with the material of the sheath shifting and creating pressure or pinch points.

Accordingly, it would be desirable to provide a structure for an elasticized composite, as might be used in a leg band or waist band in apparel or an absorbent article, which allows for adjustment during wear, is sufficiently robust to survive many cycles of machine washing and drying, and is aesthetically pleasing. Further, it would be desirable to provide an elasticized composite suitable for use in leg or waistbands which do not contribute to gapping or redmarking, and provide for a comfortable, low-bulk fit.

### SUMMARY OF THE INVENTION

What is claimed is an article comprising a sheath having a longitudinal length and an elastic component having a longitudinal length shorter than the longitudinal length of the sheath when the elastic component is in a relaxed state and moveably disposed within the sheath and a bond between the elastic component and the sheath at each end of the longitudinal length of the sheath, and at least one intermediate bond between the elastic component and the sheath between the longitudinal ends of the sheath component. There may be between 1 cm and 10 cm between each of the bonds, measured along the longitudinal length of the sheath component when the sheath component is fully extended.

The article may comprise two intermediate bonds between the elastic component and the sheath between the longitudinal ends of the sheath component. The intermediate bonds may be equidistant from the nearest longitudinal end of the sheath component when the sheath component is fully extended. The distance between the intermediate bonds may be the same as the distance from one of the intermediate bonds to a nearest longitudinal end of the sheath component when the sheath component is fully extended. The article may comprise no more than six intermediate bonds.

The sheath may be a component of a waistband or a legband. The article may be a diaper, training pant, adult incontinence device, or feminine hygiene product.

The article may comprise one intermediate bond between the elastic component and the sheath between the longitudinal ends of the sheath component. The intermediate bond may be equidistant from each longitudinal end of the sheath component when the sheath component is fully extended. The intermediate bond may be formed by sewing the elastic component to each of a front and back side of the sheath. The intermediate bond may be straight-stitched, or may be formed by a method selected from the group consisting of interfacing, adhesives, melt-welding, ultrasonic bonding, mechanical bonding, co-extrusion, mechanical fasteners, and combinations thereof.

Each intermediate bond may be placed near a natural pinch point. At least one intermediate bond may be positioned to correspond to the front of the wearer's thigh.

### BRIEF DESCRIPTION OF THE DRAWINGS

FIG. 1 is a schematic side view of an exemplary elastic structure for a disposable article.

FIG. 2A is a schematic side view of an exemplary elastic structure for a reusable article.

FIG. 2B is a perspective view of the elastic structure of FIG. 2A.

FIG. 3A is a schematic side view of another exemplary elastic structure for a reusable article.

FIG. 3B is a perspective view of the elastic structure of FIG. 3A.

FIG. 4 is a perspective view of an absorbent article, as it would be configured during wear.

FIG. 5A is a detailed view of an exemplary leg elastic structure, as shown in FIG. 4.

FIG. 5B is a detailed view of the leg elastic structure of FIG. 4 when extended.

FIG. 5C is a detailed view of an exemplary leg elastic structure having an intermediate bond.

FIG. 5D is a detailed view of another exemplary leg elastic structure having an intermediate bond.

FIG. 5E is a detailed view of an exemplary leg elastic structure having two intermediate bonds.

### DETAILED DESCRIPTION OF THE INVENTION

The term “disposable,” as used herein in reference to absorbent articles, means that the absorbent articles are generally not intended to be laundered or otherwise restored or reused as absorbent articles (i.e., they are intended to be discarded after a single use and, preferably, to be recycled, composted or otherwise discarded in an environmentally compatible manner).

The term “absorbent article” as used herein refers to devices which absorb and contain body exudates and, more specifically, refers to devices which are placed against or in proximity to the body of the wearer to absorb and contain the various exudates discharged from the body. Exemplary absorbent articles include diapers, training pants, pull-on pant-type diapers (i.e., a diaper having a pre-formed waist opening and leg openings such as illustrated in U.S. Pat. No. 6,120,487), refastenable diapers or pant-type diapers, incontinence briefs and undergarments, diaper holders and liners, feminine hygiene garments such as sanitary napkins and panty liners, and the like. Absorbent articles include reusable absorbent articles.

The term “reusable,” as used herein means that a referenced material, component, or all of an absorbent article is configured to be restored and/or reused for more than one usage cycle (e.g., a diaper change). In some reusable absorbent articles, part, or parts, or substantially all, or all of the articles may be launderable or laundering resistant, as defined and described herein. Another part or parts of a reusable absorbent article may not be launderable or laundering resistant. For example, some parts of a reusable absorbent article may be discarded after soiling and replaced with a new part, such as an absorbent insert which is used with a reusable outer cover.

The term “launderable,” as used herein means that a referenced material, component, or all of an absorbent article is configured to withstand a large number (e.g. at least 10, in some embodiments up to 50, in other embodiments more than 50) of cycles of machine washing and machine drying (as defined by AATCC Test Method 124-2001, with modifications as described below), without significant degradation to the appearance or performance of the article that would render it unsuitable for its intended functionality or use. Since hand-washing and line-drying are typically less stressful on an absorbent article than machine washing and machine drying, it is expected that a material, component, or article that is machine washable and machine dryable should also be hand-washable and hand-dryable for at least as many cycles. As an example, a reusable absorbent article may include an outer cover that is launderable. Launderable articles are designed to be suitable for use after many washings, similar to types of clothing.

The term “laundering resistant,” as used herein means that a referenced material, or component, or all of an absorbent article is configured to withstand a small number (e.g. at least one, in some embodiments up to five, in other embodiments more than five) of cycles of machine washing and machine drying (as defined by AATCC Test Method 124-2001, with modifications as described below), without significant degradation to the appearance or performance of the article that would render it unsuitable for its intended functionality and/or use. As an example, a reusable absorbent article may include an outer cover that is laundering resistant. Laundering resistant articles generally experience degradation after fewer laundering cycles than launderable articles.

The term “extensible” as used herein refers to the property of a material that elongates, without substantial rupture or breakage, by at least 20% at a load of between 0.05 and 10 N/cm in the Hysteresis Test (as described herein). Micro-sized rupture or breakage of a material is not considered substantial rupture or breakage. However, macro-sized ruptures through the structure (e.g. one or more large tears such as tears greater than about 5 millimeters in any direction, or breaking into two or more pieces, or resulting in significant structural degradation which may render the material unusable for its intended purpose) are considered substantial ruptures or breakage. A material that does not meet this definition for “extensible” is considered “inextensible.” An extensible material may be elastic or inelastic as defined herein.

The term “elastic” as used herein refers to the property of a material that elongates, without substantial rupture or breakage, by at least 20% at a load of between 0.05 and 10 N/cm in the Hysteresis Test. Further, the elastic material has a set less than or equal to 20% of the extension as measured according to the Hysteresis Test. For example, an elastic material that has an initial length of 25 millimeters can elongate to at least 37.5 millimeters (50% elongation) and, upon removal of the force, retract to a length of 27.5 millimeters, i.e., have a set of 2.5 millimeters (20% set), when subjected to the Hysteresis Test. It is to be understood, however, that this definition of elastic does not apply to materials such as individual elastic strands that do not have the proper dimensions (e.g., not wide enough) to be properly subjected to the Hysteresis Test. Instead, such material is considered to be elastic if it can elongate by at least 50% upon application of a biasing force, and return substantially to its original length (i.e., exhibit less than 20% set) upon release of the biasing force.

The term “inelastic” as used herein refers to the property of a material that elongates, without substantial rupture or breakage, by at least 20% at a load of between 0.05 and 10 N/cm in the Hysteresis Test. Further, the inelastic material has a set greater than 20% of the extension as measured according to the Hysteresis Test. For example, an inelastic material that has an initial length of 25 millimeters can elongate at least to 37.5 millimeters (50% elongation) and, upon removal of the applied force, retract to a length of 35 millimeters, i.e., have a set of 10 millimeters (80% set), when subjected to the Hysteresis Test.

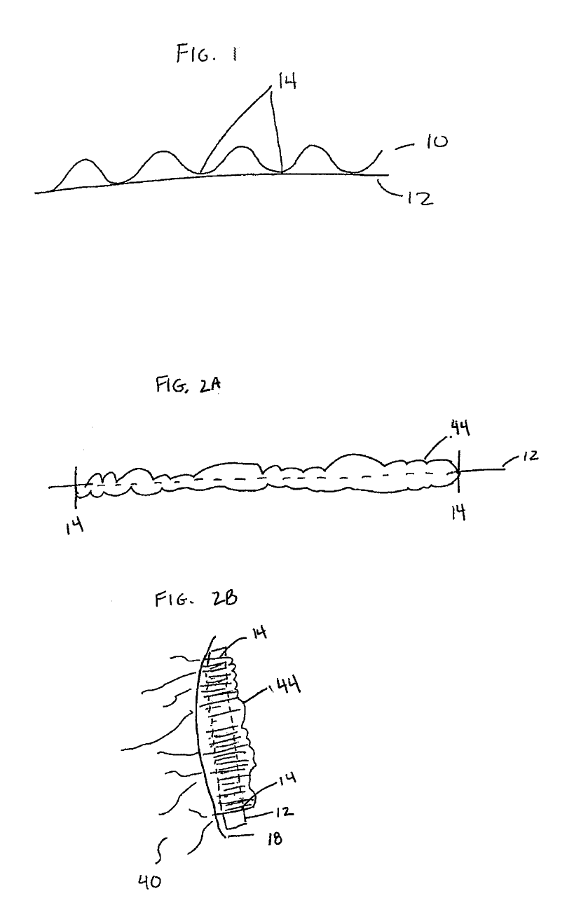

FIG. 1 shows an elastic composite material that may be used in disposable absorbent articles. Component 10, which may be elastic or inelastic, is spot-bonded to elastic component 12 at bond points 14 while elastic component 12 is in an extended state. When elastic component 12 is relaxed (that is, when elastic component 12 is allowed to contract), component 10 forms gathers as shown in FIG. 1. Elastic component 12 may be placed between two non-elastic components, and therefore, not easily visible in the finished disposable absorbent article. However, elastic component 12 is not fully enclosed in another material. Accordingly, bond points 14 must be relatively closely spaced to hold elastic component 12 in its intended location and to ensure that adequate stretchability is imparted to the composite including non-elastic component 12. Such a construction may be unsuitable for a reusable absorbent article, both because it may not be sufficiently robust to survive multiple cycles of machine washing and because the overall appearance may not be acceptable for a reusable absorbent article.

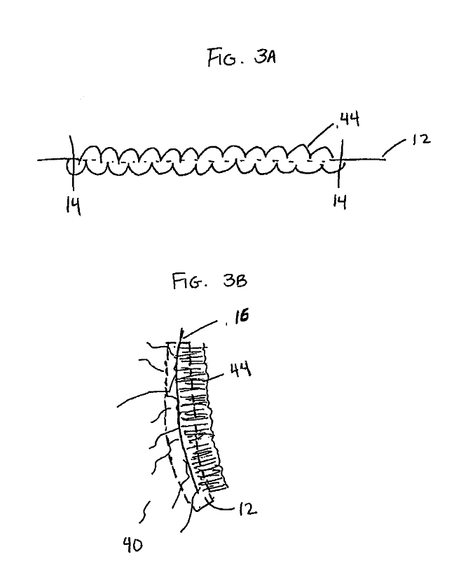

FIGS. 2A, 2B, 3A, and 3B show an elastic composite material that may be used with reusable apparel. FIGS. 2A and 2B show elastic component 12 fully enclosed in sheath component 10. Elastic component 12 may be joined to sheath component 44 at bond points 14 when elastic component 12 is in a relaxed state, such that sheath component 44 is gathered about the length of elastic component 12. Thus, the composite of sheath component 44 and elastic component 12 is elastically extensible even if sheath component 44 is not elastically extensible, because sheath component 44 will “stretch” as the gathers unfold when pulled longitudinally, and elastic component 12 will provide a contracting or return force when the longitudinal pull force is released. In FIGS. 2A and 2B, elastic component 12 is not bonded to sheath component 44 other than at bond points 14 near the longitudinal ends of sheath component 44. Between bond points 14, elastic component 12 and sheath component 44 can move relative to one another. This construction is relatively easy to make because there are only two bond points 14, however, the movement of sheath component 44 relative to elastic component 12 may lead to bunching of sheath component 44 in localized concentrations along the longitudinal length of the composite. Excessive bunching may create pinch points, where skin can get caught between folds of sheath component 44. Excessive bunching may also create regions having a high frequency of thick gathers, creating a stiff three-dimensional structure that concentrates pressure on the wearer's skin, contributing to skin indentation, chafing, marking, and erythema. Further, excessive bunching may inhibit localized stretch at other points along the longitudinal length of the composite, contributing to gapping and leakage, and may be unattractive.

In FIGS. 3A and 3B, elastic component 12 is partially enclosed in sheath component 44. Elastic component 12 is further partially enclosed by the main body 40 of the article. Overall, elastic component 12 is fully enclosed and is not directly visible in the finished article. FIG. 3B shows continuous bond 16 running through the lateral width of elastic component 12. Continuous bond 16 may be the bond connecting leg band 28 or waist band 26 to main body 40 of the article. For example, continuous bond 16 may be a stitched seam connecting leg band 28 to main body 40 of a reusable absorbent article. In this manner, the gathering of both sheath component 44 and main body 40 can be planned and controlled, and the gathering that is possible in the construction of FIG. 2B is avoided. This construction requires greater precision during fabrication to maintain the position of elastic component 10 relative to both non-elastic component 12 and main body 40 of the article, and may therefore be more time-consuming and costly to manufacture. Further, it may be desirable to allow for some movement of elastic component 12 relative to sheath component 44 and main body 40 of the article.

It has been found that providing one or more intermediate bonds 36 between bond points 14 can provide an improved appearance, more homogenous rugosities for improved skin health, comfort, and leakage, and better force distribution relative to the elastic composite constructions exemplified in FIGS. 1-3B. Specifically, allowing the sheath to gather in controlled increments may help prevent red-marking of the skin and redistribute tension along the longitudinal length of the composite. By redistributing tension, intermediate bonds 36 may reduce gapping along the composite in an article when the article is applied to a wearer. Reduced gapping, or better conformance to the body of a wearer, may be preferred aesthetically and, in absorbent articles, may help reduce the leakage of exudates from the absorbent article during use.

FIG. 4 shows absorbent article 20 having elasticized leg band 28 and elasticized waist band 26. Waist band 26 extends along the circumference of waist opening 22, and leg band 28 extends along the circumference of leg opening 24. Leg band 28 and waist band 26 may have inactive regions of elastic 30. Inactive regions of elastic 30 may be helpful in assembling the final article by providing untensioned portions of elastic component 12 which can be easily attached to other components of absorbent article 20. Inactive regions of elastic 30 may be formed, for example, by placing bond points 14 such that regions of elastic component 12 which extend beyond bond points 14 are in a relaxed state and are enclosed by a material at its full extension, such that the elastic is untensioned and another layer of material prevents extending or tensioning the elastic to any significant degree.

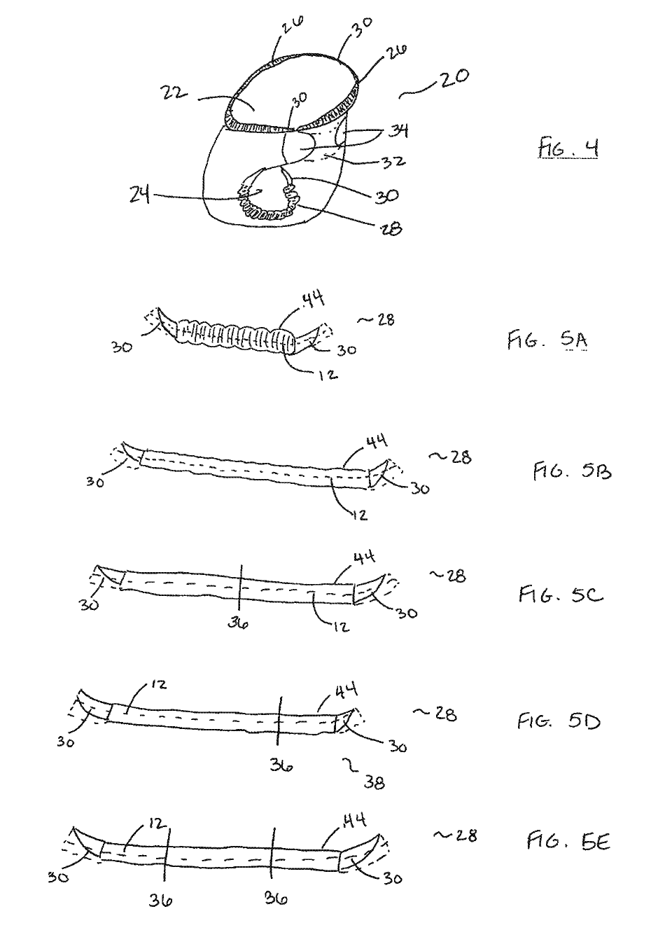

FIG. 5A shows leg band 28 isolated from absorbent article 20. Elastic component 12 is fully enclosed in sheath component 44, with inactive regions of elastic 30 at each longitudinal end. The embodiment shown in FIG. 5A may be formed by forming bond points 14 when elastic component 12 is extended, and allowing sheath component 44 to gather when elastic component 12 is relaxed. Alternately, bond points 14 may be formed when elastic component 12 is relaxed, with sheath component 44 gathered along elastic component 12. In either assembly, leg band 28 is elastically extensible, regardless of whether sheath material 44 is extensible, as shown in FIG. 5B.

In some embodiments, there may be only one intermediate bond 36 between elastic component 12 and sheath component 44 along the longitudinal length of composite 42. In other embodiments, for example, for adult-sized products, there may be six or more intermediate bonds between elastic component 12 and sheath component 44 along the longitudinal length of composite 42. The number of bonds and their placement will depend on the length of the composite, the materials used to form the composite, and the properties of the sheath component 44. For example, a softer, more pliant sheath component 44, or a combination of sheath component 10 and elastic component 12 with a relatively low coefficient of friction between the components, may not tend to form pinch points, and may, therefore, lend itself to fewer intermediate bonds 36. In contrast, a thicker or stiffer sheath component 44 may tend to form pinch points or to gather in undesirable patterns, and may, therefore, lend itself to more intermediate bonds 36.

Generally, intermediate bonds 36 should be spaced no less than 1 cm apart and no more than 10 cm apart. As the space between intermediate bonds 36 decreases below 1 cm, the benefits of allowing the sheath to gather according to the tensions present in the composite are decreased. As the space between intermediate bonds 36 increases above 10 cm apart, the benefits of controlling the sheath gathers are decreased. The distance between bonds is measured in the final product, in a flat, laid-out configuration. For measuring, the elastic components are in their as-made condition, i.e., the elastics are extended, rather than relaxed. The sheath-elastic composite is not stretched beyond the original length of the sheath fabric. That is, only elastic component 12 is extended, even if sheath component 44 is itself stretchable or elastic.

In some embodiments, intermediate bonds 36 are placed equidistant from the longitudinal ends of composite 42. Of course, variations in manufacturing processes, the materials used to make composite 42, and shifting of materials during use of composite 42 mean that the placement of intermediate bonds 36 may not be precisely equidistant from the longitudinal ends of composite 42. However, intermediate bonds 36 may be considered equidistant from the longitudinal ends of composite 42 if the distance from each end is equal within ±10% of the longitudinal length of composite 42.

In some embodiments, an intermediate bond 36 may be placed at one or more points along the composite which is particularly prone to undesirable or uneven gathering during use. For example, in an absorbent article 20 for an infant, it may be desirable to place an intermediate bond 36 along the portion of composite 42 corresponding to the front of the wearer's thigh during use. An intermediate bond 36 placed to control tension and/or gathering at a particular location may be used with other intermediate bonds 36. When additional intermediate bonds 36 are used, they may be equidistant from the longitudinal ends of composite 42, or they may be spaced differently due to the presence of an intermediate bond at a particular functional point.

Intermediate bond or bonds 36, like bond points 14, may be formed by any method known in the art, such as by stitching, interfacing, adhesives, melt-welding, ultrasonic bonding, mechanical bonding, co-extrusion, mechanical fasteners, and combinations thereof. Stitching may include any suitable sort of stitch, such as straight-stitching, zig-zag stitching, or stretch-stitching. If stitching is done by hand, any suitably strong and durable stitch, such as a backstitch, catchstitch, or oversewing stitch, may be used. In contrast, tacking or basting stitches may not be sufficiently durable to maintain a bond between elastic component 12 and sheath component 44 during the extensions and contractions associated with regular wear. Mechanical fasteners may include button-and-buttonhole, hook-and-loop, hook-and-eye, snap, tab-and-slot, and other fasteners known to produce a durable bond or connection between different materials.

An intermediate bond 36 may bond elastic component 12 to one side of the sheath formed by sheath component 44, or an intermediate bond 36 may bond elastic component 12 to both sides of the sheath formed by sheath component 44. For example, elastic component 12 may be stitched to one part of sheath component 44 before sheath component 44 is folded over itself to form a sheath, or elastic component 12 may be stitched to both parts of sheath component 44 after the sheath is formed. In some embodiments, intermediate bond 36 may overlap or be part of continuous bond 16 or main body 40 of the article. For example, intermediate bond 36 may be a stitch which extends through the entire lateral width of sheath component 44, the entire lateral width of elastic component 12, and laterally through part or all of continuous bond 16 or seam 18.

Suitable elastic components include, but are not limited to woven, knitted, braided, or extruded elastic bands, tapes, webbings, braids, laces, cords, and the like.

Suitable launderable sheath components include, but are not limited to cotton, wool, bamboo, hemp, silk, rayon, as well as blends of these materials with synthetic fibers, polyester, nylon, Lycra, Spandex, or other elastomers; breathable waterproof materials with microscopic pores smaller than a water droplet but larger than a water vapor molecule, such as GORE-TEX® (W. L. Gore & Associates, Inc., Elkton, Md.); fabrics comprising microencapsulated phase-change polymer materials such as Outlast ComforTemp fabrics (Outlast Technologies, Boulder, Colo.—see U.S. Pat. No. 6,514,362 and U.S. Pat. No. 6,207,738, for example); fiber-based moisture wicking systems, such as COOLMAX® (INVISTA, Wichita, Kans.); and the like. These materials preferably include at least one fiber-based material, such as a fabric or woven or nonwoven web. However, outer cover 10 may additionally comprise a film layer to provide enhanced liquid penetration resistance and/or elastic properties to the outer cover. Elastic properties can be added or enhanced via the addition of other materials to the outer cover, including elastic strands, bands, scrims, and the like.

Launderable materials may be formed in any known weave or fabric form, including birdseye fabric; terry; fleece; flannel; knits; stretch knits; sherpa; suedecloth; microfleece; satin; velour; Burley knits; and a dual-surface, tight-construction fabric such as Polartec® Windpro® (Polartec, LLC, Lawrence, Mass.). Knitted textiles, which may be more inherently stretchable and elastic than woven or nonwoven materials, may impart better fit, comfort and/or appearance to the outer cover. Incorporation of fibers of spandex or other elastomer also may also enhance stretchability and elasticity, and thereby impart better fit, comfort and/or appearance to the outer cover, than textiles not including such elastomeric fibers. Specific suitable examples for launderable outer cover materials include, but are not limited to, jersey knits of blends of: rayon (93%) and spandex (7%) fibers; modal (94%) and spandex (6%) fibers; cotton and spandex fibers; and bamboo and spandex fibers. Launderable materials may have basis weights of about 0.09-0.15 gram/in.² per layer, or other basis weights (basis weight may be determined using EDANA/INDA method WSP 130.1 (05), except in step 4(b), use a sample size of 25 mm×20 mm, and disregard step 5.2).

Outer covers that are laundering resistant may be sufficiently inexpensive to allow them to be discarded without issues of cost or conscience if soiled extensively or damaged, while still providing some benefit in terms of reducing environmental impact from product disposal. Laundering resistant outer cover materials may include any of the materials described herein, including one or more materials contemplated for use in launderable or disposable outer covers. If materials for use in launderable outer covers are selected, typically less expensive, lower quality (e.g., lower basis weight, less optimal fiber quality) versions may be employed, to form outer covers that are laundering resistant. If materials for use in disposable outer covers are selected, higher basis weights and/or quality of materials may be appropriate. Blends or laminates of such materials are also contemplated for laundering resistant outer covers.

Laundering resistant materials of which an outer cover may be constructed may include non-woven web materials of polypropylene and/or polyethylene fibers, polyester fibers, and any other synthetic fibers used to form nonwoven web materials used as components of disposable diapers, and blends thereof. Natural fibers such as cotton, linen, wool, bamboo, hemp, silk, rayon, and the like may be blended with synthetic fibers to form such a nonwoven web suitable as a component layer of an outer cover. An outer cover according to the present disclosure may further include films in at least some areas, as, for example, films of polypropylene and/or polyethylene.

Non-limiting examples of fibers, nonwovens and laminates of nonwovens and films that might be considered for use as laundering resistant outer cover materials may be found in U.S. Pat. Nos. 7,223,818; 7,211,531; 7,060,149; 6,964,720; 6,905,987; 6,890,872; 6,884,494; 6,878,647; and 5,518,801; and U.S. Published Applications Nos. 2008/0319407; 2008/0045917; 2007/0293111; 2007/0287983; 2007/0287348; 2007/0249254; 2007/0203301; and 2005/0164587.

### Test Methods

### Modified AATCC Test Method 124-2001

In a wash cycle, a component of an absorbent article, such as an outer cover, is machine washed and machine dried according to the protocol from AATCC (American Association of Textile Chemists and Colorists) Test Method 124-2001, with the selected parameters and variations listed below.

a) Per section 6, Apparatus and materials, a Kenmore 600 (Heavy Duty—Super Capacity Plus—Quiet Pak) is used for the automatic washing machine, and a Maytag Commercial (such as model numbers MDE27MNACW, MDE15MNAYW, and MDE13MNACW) is used for the automatic tumble dryer.

b) Despite the instructions in Section 6, Apparatus and materials, the following ballast is used: Test Fabric style 493 from Testfabrics, Inc, West Pittston, Pa., which is cotton sheeting, with a thread count of 60×60, a weight of 151 gsm, and a size of 55′ by 39″.

c) Despite the instructions in Section 6, Apparatus and materials, the evaluation area is not configured according to section 6.7 and the apparatus of section 6.8 is not used. Instead, all visual evaluations are performed under typical artificial lighting conditions (e.g. fluorescent light), which allows a person with normal vision to clearly see.

d) Despite the instructions in Section 7, Test Specimen, the component to be tested is (as necessary) entirely removed from the rest of the absorbent article, and (to the extent allowed by the removal) the component is tested as an undamaged whole. Up to three components of the same type are washed simultaneously.

e) Regarding the machine wash in Section 8.2.2, use the “large” setting on the machine for the water level, select a wash temperature of 32+/−3° C. (90+/−5° F.), and a rinse temperature of 16+/−3° C. (60+/−5° F.).

f) Regarding the settings in Section 8.2.2, select Normal/Cotton Sturdy, which has a washing time of 12 minutes, an initial spin time of 6 minutes, a refill time of 4 minutes, a rinse time of 5 minutes, and a final spin cycle time of 6 minutes.

g) Regarding the Drying in Section 8.3, select Cotton Sturdy and Whites & Colors.

h) Despite the instructions in Section 8.5, the steps of conditioning and preconditioning are not performed.

i) Despite the instructions in Section 9, Evaluation, these evaluation steps are not performed. Instead, the tested component is evaluated by one of skill in the art, to determine whether the testing has resulted in significant degradation to the appearance or performance of the article that would render it unsuitable for its intended functionality and/or use.

### Simple Tensile Test for Force Wall

The materials of this invention when pulled in a Tensile Test show two distinct regions: the first region in which the main contribution to the force is from the elastic portion of the laminate and a second region in which the force contribution from the inelastic portion of the laminate starts becoming significant. In the force versus strain curve, the second region has a significantly higher slope compared to the first region. The percent strain at which this change in slope occurs is referred to as the Force Wall.

The Simple Tensile Test for Force Wall and also the Hysteresis Test to follow utilize a commercial tensile tester (e.g., from Instron Engineering Corp. (Canton, Mass.), SINTECH-MTS Systems Corporation (Eden Prairie, Minn.) or equivalent) interfaced with a computer. The computer is used to control the test speed and other test parameters and for collecting, calculating, and reporting the data. The tests are performed under laboratory conditions of 23° C.±2° C. and relative humidity of 50%±2%. The samples are conditioned for 24 hours prior to testing.

### Test Protocol

1. Select a sample that is 7.62 cm long and 1.5 cm wide, with the long dimension being in the direction of stretch. If a waist or leg band sample is taken from a product, the width of the sample should be the entire width of the waist or the leg band. In some cases, if it is not be possible to get a 7.62 cm long sample, a smaller sample may be used, but a gage length of 25 mm must still be used.
2. Select the appropriate jaws and load cell. The jaws must have flat surfaces and must be wide enough to fit the sample (e.g., at least 2.54 cm wide). Also, the jaws should provide adequate force to ensure that the sample does not slip during testing. The load cell is selected so that the tensile response from the sample tested is between 25% and 75% of the capacity of the load cell used.
3. Calibrate the tester according to the manufacturer's instructions.
4. Set the distance between the grips at 25 mm.
5. Place the sample in the flat surface of the jaws such that the longitudinal axis of the sample is substantially parallel to the gauge length direction. Mount the sample with minimal slack. Set the slack preload at 0.02 N/cm. This means that the data collection starts when the slack is removed with a force of 0.02 N/cm. Strain is calculated based on the adjusted gauge length (lᵢₙᵢ), which is the length of the sample in between the grips of the tensile tester at a force of 0.02 N/cm. This adjusted gauge length is taken as the initial sample length, and it corresponds to a strain of 0%. Percent strain at any point in the test is defined as the change in length divided by the adjusted gauge length times 100%.
6. Pull the sample at a constant cross head speed of 254 mm/min to 1000% strain (i.e., 11× the lᵢₙᵢ), or until the sample breaks.
7. The computer records the force exerted on the sample during the test as a function of applied strain.
8. Plot force (N/cm) versus percent strain and measure the force wall as the strain at which the slope of the force vs. strain curve increases sharply. This can either be done manually or using the software of the tensile tester.
9. Perform 5 repetitions on each sample and report average and standard deviation.

### Hysteresis Test

Steps 1 to 5 are same as for the Force Wall Test.

6(a). First cycle loading: Pull the sample to the force wall at a constant cross head speed of 254 mm/min. The force wall is determined first as described in the Simple Tensile Test above. At the force wall, report the stretched sample length between the jaws as lₘₐₓ.

6(b). First cycle unloading: Hold the sample at the force wall strain for 30 seconds and then return the crosshead to its starting position (0% strain) at a constant cross head speed of 254 mm/min. Hold the sample in the unstrained state for 1 minute.

6(c). Second cycle loading: Pull the sample to the force wall at a constant cross head speed of 254 mm/min.

6(d). Second cycle unload: Next, return the crosshead to its starting position (i.e. 0% strain) at a constant cross head speed of 254 mm/min.

A computer data system records the force exerted on the sample during the test as a function of applied strain. From the resulting data generated, the following quantities are reported (note that loads are reported as force divided by the width of the sample and do not take into account the thickness of the sample):
1. First cycle load force and percent strain at the force wall (N/cm).
2. First cycle unload force at a strain that is 15% of the strain at the force wall. (For example, if the force wall is at 200%, unload is measured at 30% strain).
3. % set: Record length of sample at a second cycle load force of 0.02 N/cm (lₑₓₜ). Calculate % set as the strain measured at a second cycle load of 0.02N/cm reported as a percent of the strain at the force wall)

% set=(lₑₓₜ−lᵢₙᵢ)/(lₘₐₓ−lᵢₙᵢ)*100%.

Five repetitions are done on each sample and the average and standard deviation reported.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An article comprising
a sheath having a longitudinal length; and
an elastic component having a longitudinal length shorter than the longitudinal length of the sheath when the elastic component is in a relaxed state and moveably disposed within the sheath; and
a bond between the elastic component and the sheath at each end of the longitudinal length of the sheath, and at least one intermediate bond between the elastic component and the sheath between the longitudinal ends of the sheath component.

2. The article of claim 1, wherein there is between 1 cm and 10 cm between each of the bonds, measured along the longitudinal length of the sheath component when the sheath component is fully extended.

3. The article of claim 1, comprising two intermediate bonds between the elastic component and the sheath between the longitudinal ends of the sheath component.

4. The article of claim 3, wherein the intermediate bonds are equidistant from the nearest longitudinal end of the sheath component when the sheath component is fully extended.

5. The article of claim 4, wherein the distance between the intermediate bonds is the same as the distance from one of the intermediate bonds to a nearest longitudinal end of the sheath component when the sheath component is fully extended.

6. The article of claim 1, comprising no more than six intermediate bonds.

7. The article of claim 1, wherein the sheath is a component of a waistband.

8. The article of claim 1, wherein the sheath is a component of a legband.

9. The article of claim 1, wherein the article is selected from the group consisting of diapers, training pants, adult incontinence devices, and feminine hygiene products.

10. The article of claim 1, comprising one intermediate bond between the elastic component and the sheath between the longitudinal ends of the sheath component.

11. The article of claim 10, wherein the intermediate bond is equidistant from each longitudinal end of the sheath component when the sheath component is fully extended

12. The article of claim 1, wherein the intermediate bond is formed by sewing the elastic component to each of a front and back side of the sheath.

13. The article of claim 12, wherein the intermediate bond is straight-stitched.

14. The article of claim 1, wherein each bond is formed by a method selected from the group consisting of interfacing, adhesives, melt-welding, ultrasonic bonding, mechanical bonding, co-extrusion, mechanical fasteners, and combinations thereof.

15. The article of claim 1, wherein each intermediate bond is placed near a natural pinch point.

16. The article of claim 15, wherein at least one intermediate bond is positioned to correspond to the front of the wearer's thigh.
